(19) United States
(12) Patent Application Publication
(10) Pub. No.: US 2014/0066869 A1
(43) Pub. Date: Mar. 6, 2014

(54) DEODORISING COMPOSITION

(71) Applicant: John Toft, Stockport (GB)

(72) Inventor: John Toft, Stockport (GB)

(73) Assignee: GX LABS HOLDINGS LIMITED, London (GB)

(21) Appl. No.: 14/114,125

(22) PCT Filed: Apr. 24, 2012
(86) PCT No.: PCT/GB2012/050903

§ 371 Date: Oct. 25, 2013

(30) Foreign Application Priority Data
Apr. 28, 2011 (GB) 1107181.8

(51) Int. Cl. A61F 13/84; A61K 8/97; A61K 8/37; A61Q 15/00; A61K 8/41

(52) U.S. Cl. CPC A61F 13/8405; A61Q 15/00; A61K 8/416; A61K 8/37; A61K 8/97
USPC 604/359; 424/76.8

(57) ABSTRACT

The present invention relates to an absorbent article, such as a sanitary towel, impregnated or coated with a deodorizing composition comprising a buffering component, a deodorizing agent, and an antimicrobial agent. Such deodorizing compositions help to reduce or eliminate odours emanating from the bodily fluids absorbed into the article during use.

### INTRODUCTION

The present invention relates to an absorbent article, such as a sanitary towel, nappy, or incontinence pad, which is configured to absorb and retain secreted or excreted bodily fluids. More particularly, the present invention relates to an absorbent article comprising a deodorizing composition for reducing or eliminating odours that would otherwise emanate from the secreted or excreted bodily fluids absorbed in the by the article during use. The invention also relates to a deodorizing composition per se, to a method of manufacturing the deodorizing composition, to a method of applying the deodorizing composition to an article, and to the use of the deodorizing composition in or on an article to reduce or eliminate odours resulting from secretion or excretion of bodily fluids.

### BACKGROUND OF THE INVENTION

Articles designed to absorb and retain bodily fluids, such as sanitary towels, nappies, and incontinence pads, are widely used. However, with all of these articles there is a need to reduce and/or eliminate the odours that can emanate from the captured bodily fluids or the by-products thereof (e.g. volatile malodorous compounds resulting from the microbial degradation of bodily fluids). This is especially the case for articles such as sanitary towels, which retain bodily fluids for prolonged periods in an environment which is conducive to microbial growth (i.e. moist environment and warm temperature).

In the case of sanitary towels, fragranced formulations are in current usage which, when applied to the sanitary towel, seek to mask any odours that may be present. Unfortunately, however, such fragrances often fail to mask strong odours, and also produce a characteristic odour of their own, which may be undesirable.

Moreover, women are advised to replace sanitary towels at regular intervals for reasons of comfort (i.e. reduced irritation) and to reduce the risk of odour formation and emanation. However, regularly changing sanitary towels is not always convenient, so the provision of sanitary towels comprising a suitable deodorizing composition would be a distinct advantage.

It is therefore an object of the present invention to solve at least some of the abovementioned problems.

### SUMMARY OF THE INVENTION

In a first aspect of the present invention there is provided an absorbent article configured to absorb and retain bodily fluids (e.g. menstrual fluids, urine, blood or sweat) during use, wherein the article is coated or impregnated with a deodorizing composition comprising a buffering component, a deodorizing agent, and an antimicrobial agent. The deodorizing composition reduces or eliminates odours emanating from the bodily fluids absorbed into the article during use.

In a second aspect of the present invention there is provided a deodorizing composition comprising a buffering component, a deodorizing agent, and an antimicrobial agent. The deodorizing composition is suitable for application to an absorbent article as defined herein.

In a third aspect of the present invention there is provided a method of manufacturing the deodorizing composition of the second aspect (in liquid form) the method comprising mixing a buffering component, a deodorizing agent and an antimicrobial agent in a suitable diluent; and optionally adjusting the pH of the composition. Compositions in a dried form can be prepared by drying the liquid form of the composition to remove the diluent.

In a fourth aspect of the present invention there is provided a method of forming an absorbent article of the first aspect of the invention, the method comprising coating or impregnating the absorbent article or a component part thereof with a deodorizing composition as defined herein. If the deodorizing composition is in a liquid form method may optionally comprise the subsequent step of drying the article.

In a fifth aspect of the present invention there is provided the use of the deodorizing composition (of the second aspect) in or on an absorbent article to reduce or eliminate odours resulting from secreted or excreted bodily fluids.

Advantageously, absorbent articles of the present invention are particularly beneficial at neutralizing odours arising from bodily fluids that have been captured and/or retained within the article during use. The articles of the present invention are particularly useful for neutralizing strong and unpleasant odours. The buffering component of the composition can advantageously assists in the neutralization of certain odour forming chemicals. For example, when the deodorizing composition disperses within the bodily fluid present within the article, it typically provides a buffered environment with an acidic pH, which can help neutralize odours caused by volatile and non volatile basic amine compounds. Moreover, the growth of microbes responsible for generating certain unpleasant odours upon digestion of bodily fluids within the articles of the present invention is substantially reduced or eliminated by the use of an antimicrobial agent. Additionally, the ingredients present within the deodorizing composition can be selected so as render the composition, and any absorbent article of the invention to which the deodorizing composition is applied, substantially non-irritant. For example, nappies, sanitary towels etc. according to the invention are all substantially non-irritant to the skin, even over prolonged use. This permits articles such as sanitary towels, nappies etc. to be replaced less frequently and to be worn in comfort and with substantially reduced or eliminated odour problems.

In addition, articles of the present invention also do not substantially affect the natural flora on the skin of the user, since any antimicrobial activity is generally mild and localised within the article itself.

The absorbent articles of the present invention are also simple and cheap to produce, merely requiring the addition of the deodorizing composition to appropriate parts of the article. As such, the deodorizing compositions of the present invention can be readily applied to existing untreated articles either during or after their manufacture, or even by the end-user before prior to the use of the article. In the latter case, the deodorizing compositions of the present invention can be conveniently provided in a “ready-to-use” form for easy application to the absorbent article by the user.

### BRIEF DESCRIPTION OF THE DRAWINGS

FIG. 1 is a bar chart showing a data summary for scoring odour for different bacteria and yeast inoculated pads either treated or untreated with 600 ppm of the GX deodorizing composition (20% w/v ethanolic solution of the undiluted deodorizing composition of Example 1) after incubation for 0 or 6 hours at 35° C.+/−1° C.

### DETAILED DESCRIPTION OF THE INVENTION

### Definitions

Unless otherwise stated, the following terms used in the specification and claims have the following meanings set out below.

Herein, the term “bodily fluids” is intended to refer to a fluid released, e.g. secreted or excreted, by the human or animal body. Such fluids typically include menstrual fluids, urine, sweat, and the like.

Herein, the term “buffering component” is used to refer one or more components that make up a buffer system capable of regulating the pH of any bodily fluids absorbed into an absorbent article during use. Suitably the buffering component maintains the pH environment within a physiologically acceptable pH range. Suitable buffering components will be readily understood by those skilled in the art, and will typically consisting of a mixture of a weak acid and its conjugate base, or alternatively a weak base and its conjugate acid. However, it will be understood also that an appropriate acid may on its own (i.e. without any conjugate base) suffice as the buffering component, for instance where lactic acid is used as the buffering component.

Herein, the term “deodorizing” is intended to mean reducing and/or eliminating detectable odours. This absence of detectable odours can be assessed simply by smell or by other analytical techniques to detect the presence of particular known odour causing chemicals. Such analytical techniques may be conducted using, for example, spectroscopy (e.g. ultraviolet, infra-red, nuclear magnetic resonance) or mass spectrometry techniques well known in the art, or a mixture of such techniques, optionally in conjunction with chromatography (e.g. LCMS, GCMS).

Herein, a “diluent” is intended to mean a solvent or vehicle for the components of the deodorizing composition. The diluent is typically a protic solvent such as alcohol or water.

Herein, a weight percent (wt %) refers to the percentage by weight in relation to the whole of the relevant material, be it an article or a composition.

### The Article

The present invention provides an absorbent article configured to absorb and retain bodily fluids (e.g. menstrual fluids, blood or sweat) during use, wherein the article is coated or impregnated with a deodorizing composition comprising a buffering component, a deodorizing agent, and an antimicrobial agent.

The deodorizing composition reduces or eliminates any odours emanating from the bodily fluids absorbed into the article during use.

The deodorizing composition is suitably coated upon or impregnated within the article of the present invention by applying the composition to the appropriate part of the article. The composition may be applied in a dried form (e.g. as a powder), or as a concentrate (typically a liquid concentrate) which may be essentially self-drying. Alternatively, the composition may be applied to the article in a liquid form, which is then dried in situ so as to provide an absorbent article comprising a dried deodorant composition. The dried composition then dissolves in the presence of bodily fluids.

In its dry form, the composition resides in the article, optionally as a solid dispersion throughout the article, or a portion thereof, or as a film or coating within the article, or a portion thereof.

The article is preferably an article for use on, or in close proximity to, the human body, preferably such that, during use, the article receives and retains secreted or excreted bodily fluid. In a particular embodiment the article comprises a retention zone to receive and retain any bodily fluids absorbed by the article.

In a particular embodiment, the article is in direct contact with the human body, i.e. adjacent to the skin.

The article may be selected from the group comprising sanitary items (e.g. sanitary towels, tampons), incontinence pads, nappies, sweatbands, items of clothing (e.g. underwear), footwear (e.g. the soles of shoes, slippers, boots, trainers etc.), headgear (e.g. hats or helmets); linen (e.g. bed linen); towels or medical fabrics (e.g. bandages, wound dressings).

In an embodiment, the article is suitably a hygiene product, such as a nappy, sanitary towel, maternity pad or incontinence pad. In a particular embodiment the article is a sanitary towel.

In many instances, the article will be disposable, for example, it will be disposed of after a single use.

Typical absorbent articles of the present invention, such as a nappy, sanitary towel, maternity pad or incontinence pad, are impregnated and/or coated with the deodorizing composition on at least a part or portion of the article. Such articles typically comprise an absorbent/retaining layer or zone, which receives and retains the bodily fluid within the article, and, optionally, a separate acquisition layer or zone through which the bodily fluids flow as they are absorbed into the absorbent/retaining layer or zone of the article. Typically, the acquisition layer or zone will not retain any bodily fluids.

The deodorizing composition may suitably be present in an absorbent/retaining zone of the article and/or in the acquisition layer or zone.

For example, a sanitary towel will typically comprise an absorbent core (the absorbent/retaining zone), which is made from a suitably absorbent material, preferably a superabsorbent polymer and/or cellulosic fibres. The sanitary towel may additionally comprise an acquisition layer that covers at least one surface of the core. The acquisition layer is disposed between the absorbent core and the body of the user during use. In an embodiment, at least some of the deodorizing composition may be present in the absorbent core, and in a particular embodiment, all of the deodorizing composition is present in the absorbent core. In another embodiment, at least some of the deodorizing composition may be present in the acquisition layer (deodorizing zone), and in a particular embodiment all of the deodorizing composition is present in the acquisition layer. In a particular embodiment the deodorizing composition is present on an underside of the acquisition layer (i.e. the side facing the absorbent core, which is not in direct contact with the body during use).

The article suitably comprises a sufficient quantity of the deodorizing composition to provide the required deodorizing effect during use. In a particular embodiment, the article suitably comprises an amount of the deodorizing composition which is sufficient to act as a bacteriostat (i.e. to prevent growth of bacteria/microbes rather than kill all bacteria/microbes present). In alternative embodiment, the article suitably comprises a quantity of deodorizing composition which is sufficient to provide a bacteriocidal effect (i.e. to kill and reduce the number of bacteria/microbes present).

The article suitably comprises 0.05 to 7 wt % of the deodorizing composition herein described, suitably 1 to 6 wt %, more suitably 2 to 5 wt %, and even more suitably 3 to 4 wt %. In this context, the weight percent (wt %) of the deodorizing composition is based on the percentage dry weight (i.e. without a diluent) of the deodorizing composition relative to the total dry weight of the article.

By way of example, the article may suitably comprise sufficient deodorizing composition to provide (based on the total weight of the article):
0.05 to 5 wt % of buffering component
0.01 to 1 wt % of antimicrobial agent
0.01 to 1 wt % of deodorizing agent.

In a particular embodiment, the article comprises sufficient deodorizing composition to provide (based on the total weight of the article):
0.1 to 1 wt % of buffering component
0.05 to 0.3 wt % of antimicrobial agent
0.05 to 0.5 wt % of deodorizing agent.

In a particular example, the article (suitably a sanitary towel) suitably comprises sufficient deodorizing composition to provide (based on the total weight of the article):
0.0005 to 1 wt % of buffering component
0.01 to 1 wt % of antimicrobial agent
0.01 to 2 wt % of deodorizing agent.

In a particular embodiment, the article comprises sufficient deodorizing composition to provide (based on the total weight of the article):
0.001 to 0.05 wt % of buffering component
0.05 to 0.2 wt % of antimicrobial agent
0.1 to 1 wt % of deodorizing agent.

The article with the deodorizing composition preferably reduces odours resulting from the secretion or excretion of bodily fluids compared to the equivalent article without the deodorizing composition present.

The deodorizing composition preferably creates a localised acidic environment within the article when bodily fluids are absorbed into the article. Preferably the localised acidic environment is sufficiently acidic to react with certain volatile basic odour producing compounds that may be present, such as ammonia and/or amines. Preferably the localised acidic environment has a pH of between 3.5 and 5.5, preferably between 3.8 and 4.2, more preferably between 3.9 and 4.1, and most preferably at about pH 4.

In one aspect, the invention relates to an article, e.g. a sanitary towel, comprising a deodorizing composition and/or mixture as described in any one of the present Examples, including any of the articles described in the present Examples.

### Deodorizing Composition

The present invention provides a deodorizing composition for reducing or eliminating odours resulting from bodily fluids, the composition comprising a buffering component, a deodorizing agent, and an antimicrobial agent.

The deodorizing composition may be in a solid (e.g. powder) form or in a liquid form (e.g. dilute or concentrated).

In an embodiment, the deodorizing composition is in a liquid form and comprises the buffering component, the deodorizing agent, and the antimicrobial agent in a suitable diluent. Any suitable diluent may be used. In an embodiment, the diluent is selected from water or alcohol (e.g. ethanol). In liquid form, the deodorizing composition can be easily applied to the article, for example in the form of a concentrate, spray, dip coating, or paint. In an embodiment, the deodorizing composition is provided as a concentrate which comprises, for example, less than 80 wt % diluent or less than 70 wt % diluent in the composition. In a particular embodiment, the deodorizing composition is a liquid concentrate, suitably a concentrate spray. Such concentrates suitably require minimal or no drying following their application to the article.

The deodorizing composition may suitably comprise 30-99.5 wt % diluent, for example 50-99 wt % or 70-98 wt % diluent.

In an embodiment, the deodorizing composition is in a dried form (i.e. it is substantially diluent free). The composition may be applied to the article in a dried form or, alternatively, applied in a liquid form and then dried in situ within or on the article.

The deodorizing composition suitably comprises the buffering component, deodorizing agent, and antimicrobial agent in a weight ratio of 5-30:0.5-20:0.1-10 (buffering component:deodorizing agent:antimicrobial agent).

In a particular embodiment, the deodorizing composition suitably comprises the buffering component, deodorizing agent, and antimicrobial agent in a weight ratio of 10-20:1-10:1-3 (buffering component:deodorizing agent:antimicrobial agent).

In another embodiment, the deodorizing composition suitably comprises the buffering component, deodorizing agent, and antimicrobial agent in a weight ratio of 10-20:3-7:1.5-2 (buffering component:deodorizing agent:antimicrobial agent).

In a particular embodiment (e.g. where the article is a sanitary towel), the deodorizing composition comprises the buffering component, deodorizing agent, and antimicrobial agent in a weight ratio of 1-3:100-1000:10-400 (buffering component:deodorizing agent:antimicrobial agent). In a particular embodiment, the deodorizing composition comprises the buffering component, deodorizing agent, and antimicrobial agent in a weight ratio of 2:200-800:20-100 (buffering component:deodorizing agent:antimicrobial agent). In a particular embodiment, the deodorizing composition comprises the buffering component, deodorizing agent, and antimicrobial agent in a weight ratio of 2:200-700:40-80 (buffering component:deodorizing agent:antimicrobial agent).

Suitably the weight ratio of the buffering component to the antimicrobial agent is 20:1 to 10:3, suitably 10:1 to 5:1.

In an embodiment (e.g. where the article is a sanitary towel), the weight ratio of the buffering component to the antimicrobial agent is 10:60 to 1:300, suitably 1:60 to 2:300.

In an embodiment, the deodorizing composition further comprises an anti-irritant (i.e. an agent that reduces the potential irritancy effect of other compounds present in the composition).

In an embodiment, the deodorizing composition further comprises a phospholipid component. The phospholipid component suitably acts as anti-irriitant. The weight ratio of the buffering component to phospholipid component in the deodorizing composition is suitably between 1:10 and 5:1, suitably between 1:5 and 1:1. In an embodiment (e.g. where the article is a sanitary towel), the weight ratio of the buffering component to phospholipid component in the deodorizing composition is suitably between 1:200 and 1:1, suitably between 1:120 and 1:80.

In a particular embodiment, the deodorizing composition further comprises a preservative. Preferably the preservative provides added broad spectrum activity against micro-organisms.

In a preferred embodiment, the deodorant composition comprises a buffering component, an antimicrobial agent, a phospholipid component, a deodorizing agent, and a preservative.

In an embodiment, the deodorizing composition is in a liquid form and it comprises 0.1 to 2 wt % buffering component, suitably 0.2 to 0.7 wt %, suitably 0.3 to 0.6 wt %, based on the total weight of the deodorizing composition for application to the article.

In an embodiment, the deodorizing composition is in a liquid form and it comprises 0.1 to 0.5 wt % antimicrobial agent, suitably 0.2 to 0.4 wt %, based on the total weight of the deodorizing composition for application to the article.

In an embodiment, the deodorizing composition is in a liquid form and it comprises 0.05 to 0.5 wt % of the deodorizing agent, suitably 0.1 to 0.2 wt %, suitably 0.1 to 0.15 wt %, based on the total weight of the deodorizing composition for application to the article.

In an embodiment, the deodorizing composition is in a liquid form and it comprises 0.1-3.0 wt % phospholipid component, suitably 0.5-3.0% wt %, suitably 1.0-2.0 wt %, based on the total weight of the deodorizing composition for application to the article.

In an embodiment, the deodorizing composition is in a liquid form and it comprises 0.1 to 1.0 wt % preservative, suitably 0.3 to 0.08 wt %, more suitably 0.05 to 0.08%, based on the total weight of the deodorizing composition for application to the article.

In a particular embodiment, the deodorizing composition is in a liquid form and it comprises:
0.1-0.5 wt % of antimicrobial agent;
0.5-3.0 wt % of phospholipid component;
0.05-3.0 wt % of deodorizing agent;
0.1-1.0 wt % of preservative;
0.2-0.7 wt % of lactic acid.

In a particular embodiment, the deodorizing composition is in a liquid form and it comprises:
0.2-0.4 wt % of antimicrobial agent; (e.g. PHMB);
0.5-2.0 wt % of phospholipid component; (e.g. Colalipid C);
0.1-0.2 wt % of deodorizing agent. (e.g. Sinodur CQ);
0.3-0.5 wt % of preservative (e.g. Nipagard PO-05);
0.3-0.5 wt % of lactic acid

In an embodiment, the deodorizing composition is provided as a concentrate, comprising:
15-45 wt % antimicrobial agent (e.g. PHMB)
20-50 wt % phospholipid component (Colalipid C)
5-15 wt % deodorizing agent (e.g. Sinodur CQ)
5-15 wt % lactic acid

In an embodiment, the deodorizing composition is provided as a concentrate, comprising:
37.5 wt % antimicrobial agent (e.g. PHMB)
41.6 wt % phospholipid component (Colalipid C)
10.4 wt % deodorizing agent (e.g. Sinodur CQ)
10.4 wt % lactic acid

Such concentrates may be formed by dispersing the solid components in a reduced volume of diluent. The amount of the diluent used is preferably sufficient enable the components to be dispersed and to permit the concentrate to flow (i.e. be sufficiently fluid).

Note that these concentrate compositions are by way of example, and omit the preservative, which is optional in all compositions.

In a particular embodiment, the deodorizing composition suitably provides a pH of between 3.5 and 4.5 (preferably between 3.8 and 4.2, more preferably 3.9 and 4.1 and most preferably pH 4) within any bodily fluid absorbed into the article.

In a particular embodiment, the deodorizing composition has a pH between 3.5 and 5.5 In a particular embodiment, the deodorizing provides a pH of between 3.5 and 5.5 with any bodily fluid absorbed into the article.

In an embodiment, the deodorizing composition (suitably in a liquid form) comprises 0.05 to 2 wt % buffering component, suitably 0.1 to 1 wt %, suitably 0.15 to 0.4 wt %, based on the total weight of the deodorizing composition for application to the article.

In an embodiment, the deodorizing composition (suitably in a liquid form) comprises 1 to 35 wt % antimicrobial agent, suitably 2 to 20 wt %, suitably 3 to 9 wt % based on the total weight of the deodorizing composition for application to the article.

In an embodiment, the deodorizing composition (suitably in a liquid form) comprises 10 to 70 wt % of the deodorizing agent, suitably 20 to 40 wt %, based on the total weight of the deodorizing composition for application to the article.

In an embodiment, the deodorizing composition (suitably in a liquid form) comprises 0.1-20 wt % phospholipid component, suitably 1-15 wt %, suitably 5-15 wt %, based on the total weight of the deodorizing composition for application to the article.

In an embodiment, the deodorizing composition is provided as a concentrate, comprising:
0.05 to 2 wt % buffering component (e.g. lactic acid)
1 to 35 wt % antimicrobial agent (e.g. PHMB)
10 to 70 wt % deodorizing agent (e.g. Duoplex)
0.1 to 20 wt % phospholipid component (e.g. Colalipid C)

In one aspect the invention relates to a deodorizing composition having the ingredients and/or weight composition of any of those described in the present Examples.

In a particular embodiment, the deodorizing composition is a concentrate, comprising:
1-10 wt % antimicrobial agent (e.g. PMBH)
25-35 wt % deodorizing agent (e.g. Deoplex)
5-15 wt % phopholipid (e.g Colalipid C)
0.1-0.5 wt % Lactic Acid

In a particular embodiment, the deodorizing composition is a concentrate, comprising:
2-8 wt % PMBH
25-35 wt % Deoplex
6-12 wt % Colalipid C
0.1-0.5 wt % Lactic Acid

Suitably, any remaining balance of ingredients (up to 100 wt %) of the compositions defined herein may consist of one or more solvents (e.g. water) and optionally one or more additional additives (e.g. preservatives).

### Buffering Component

The buffering component, as described herein, typically comprises an acid, such as lactic acid, and an alkali, such as sodium hydroxide. In some embodiments, the buffering component comprises an acid (especially an organic acid), optionally with or without a conjugate base. In a particular embodiment, no conjugate base is present. The buffer component suitably provides an acidic environment within any bodily fluid absorbed into an article, suitably with a pH of 3.5 to 5.5, suitably 3.5 to 4.5, as previously defined.

In some embodiments, the acidity neutralizes certain basic odorous compounds that may be present, particularly volatile basic odorous compounds such as amines.

The buffering component dissolves within the bodily fluids to provide the required buffering effect. The acid is suitably an organic acid and preferably is an acid having a pKₐ between 2 and 5, suitably between 3.5 and 4.5, suitably between 3.7 and 4.3, suitably between 3.8 and 4.2. The acid may for example, be an acid selected from the group comprising lactic acid, formic acid, benzoic acid, glycolic acid, tartaric acid, acetic acid, citric acid, or their derivatives. Most preferably, the acid is lactic acid.

Suitably, the acid is present at 0.01 to 2 wt % of an absorbent article, suitably 0.1 to 1 wt % of the article, suitably 0.4 to 0.6 wt % of the article.

In an embodiment, the acid is present at 0.0005 to 1 wt % of an absorbent article, suitably 0.001 to 0.05 wt % of the article, suitably 0.002 to 0.005 wt % of the article, especially where the article is a sanitary towel.

### Antimicrobial Agent

The antimicrobial agent may be any suitable antimicrobial agent known in the art which is effective at inhibiting microbial.

The antimicrobial agent may induce bateriostasis (i.e. prevent growth of bacteria/microbes, rather than kill all the bacteria/microbes present).

The antimicrobial agent is suitably an antimicrobial compound that is safe for cosmetic use (i.e. suitably non-irritant and/or non-toxic).

Examples of suitable antimicrobial agents include quaternary ammonium compounds (such as those well-known in the art, e.g. alkyldimethyl benzylammonium chloride), didecyl dimethyl ammonium chloride, amphoterics, chlorhexidine gluconate, or polyhexamethylene biguanide (PHMB), or a combination thereof.

In a particular embodiment, the antimicrobial agent is selected from quaternary ammonium compounds (such as those well-known in the art, e.g. alkyldimethyl benzylammonium chloride), didecyl dimethyl ammonium chloride, amphoterics, chlorhexidine gluconate, polyhexamethylene biguanide (PHMB), or an antimicrobial metal-containing compound (e.g. titanium dioxide (TiO₂)) or a combination thereof.

In a particular embodiment, the antimicrobial agent is PHMB. PHMB is commercially available as Cosmocil™ CQ (or Vantocil TG), a 20% w/w aqueous solution of PHMB obtainable from Arch Biocides.

In another embodiment, the antimicrobial agent is an antimicrobial metal-containing compound, such as a metal oxide, suitably a transition metal oxide, most suitably titanium dioxide (TiO₂).

### Phospholipid Component

In a particular embodiment, the deodorizing composition further comprises a phospholipid component. The phospholipid component may be a phospholipid compound or a phospholipid derivative. The phospholipid component may comprise an amphoteric phospholipid compound or derivative thereof.

The phospholipid component may also possess some antimicrobial activity in certain embodiments.

The phospholipid component is suitably biocompatible.

In a particular embodiment, the phospholipid component is an anti-irritant (i.e. it reduces the irritancy effect of other compounds present).

In a particular embodiment, the phospholipid component comprises a natural or naturally derived phospholipid compound. In a particular embodiment the phospholipid component comprises a coconut phospholipid derivative.

In an alternative embodiment, the phospholipid component comprises a synthetic phospholipid, based on natural ingredients.

In a particular embodiment, the phospholipid component comprises a compound, or an acceptable salt thereof, of Formula A:

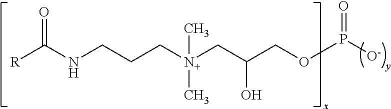

wherein x+y=3; and R is linear or branched (6-24C)alkyl, (6-24C)alkenyl, (6-24C)alkynyl. R may suitably be linear or branched (10-18C)alkyl, (10-18C)alkenyl, (10-18C)alkynyl. R preferably corresponds to the fatty acid side chain of the fatty acid from which the amide is derived.

R may suitably be selected from the group including coco (i.e. coconut fatty acid side chain (where R is (11C)alkyl), linoleyl, oleyl, stearyl, ricinoleyl, di-linoleyl, and myristyl.

In a particular embodiment, the phospholipid component comprises cocamidopropyl PG-dimonium chloride phosphate (i.e. when R of Formula A is coco ((11C)alkyl)). This compound is commercially available as Cola™ Lipid C (CAS No. 83682-78-4).

A compound of Formula A is preferably a salt. Suitably, the counterion to phosphate is an alkali metal such as sodium. Suitably the counterion to the quaternary ammonium is a halogen such as chlorine.

Herein, wherever a or any phospholipid component is mentioned, the following materials/mixtures may be direct replacements/alternatives therefor:
An amphoteric biocide
Tego™ Care CG90
Linolenic acid and/or glycerol monolaurate
Linolenic acid and/or glycerol myristate

As such, any phospholipid component disclosed herein may be duly replaced by any one of the above materials/mixtures, where relevant in the same quantities as quoted for the phospholipid component being replaced.

### Deodorizing Agent

Any suitable deodorizing agent may be used in the compositions of the present invention.

In a particular embodiment the deodorizing agent is non-fragranced and free from odour-masking agents (i.e. agents which provide a compete smell that is stronger than the odour to be masked). In a particular embodiment, the deodorizing agent is able to entrap the odour-producing compounds so as to decrease their volatility and, in preferred embodiments, this renders the odour-producing compounds substantially non-volatile. In a particular embodiment, the deodorizing agent may suitably possess a cage-like molecular structure that chelates volatile odour-producing compounds.

In a particular embodiment, the deodorizing agent comprises a deodorant compound selected from the group including methyl crotonate (e.g. Sinodur™ from Givaudan), Deoplex™ (Saccharomyces ferment by Carrubba Inc.), sodium ricinoleate (obtainable from Chemlink Specialities), zinc ricinoleate (Flexisorb™ by ICT Inc.), cyclodextrin (obtainable from Proctor & Gamble), or a combination thereof.

In a particular embodiment, the deodorizing agent comprises methyl crotonate.

### Preservative

The deodorizing composition may optionally comprise a preservative. Any suitable preservative known in the art may be used. Preferably, the preservative is suitable for cosmetic use (i.e. suitably non-irritant and/or toxic). The preservative preferably gives a broad spectrum activity against transient micro-organisms.

In a particular embodiment the preservative comprises piroctone olamine (i.e. ethanolamine salt of the hydroxamic acid). The preservative may also comprise 2-phenoxyethanol. In a particular embodiment the preservative comprises both piroctone olamine and 2-phenoxyethanol, suitably commercially available in the form of Nipaguard™ PO-05. Other preservative formulations are suitable.

### Method of Preparing the Deodorizing Composition

The present invention provides a method of manufacturing the deodorizing composition of the second aspect in a liquid form, the method comprising providing a buffering component in a diluent; mixing the diluted buffering component with a deodorizing agent and an antimicrobial agent; and optionally adjusting the pH.

Concentrate forms of the deodorizing composition may be formed by minimising the diluent, preferably by using only enough diluent to sufficiently mobilize the concentrate to allow the ingredients to be suitably mixed.

Preparing the buffering component in the diluent may comprise mixing a suitable acid with the diluent, and then adjusting the pH by adding a suitable base to yield at least some conjugate-base of the acid and a suitable counterion. Suitably the base is the oxide or hydroxide salt of the counterion. In a particular embodiment, the pH of the composition is adjusted to be at a pH of 3.5 to 5.5, suitably 3.5 to 4.5, suitably 3.8 to 4.2, more suitably 3.9 to 4.1, and most suitably pH 4. Adding the base may suitably leave a molar excess of the acid relative to the conjugate-base of the acid.

The method may comprise adding additional components as hereinbefore described (e.g. the antimicrobial agent, deodorizing agent), to the diluent either before or after preparing the buffering component in the diluent.

The method may also comprise formulating the deodorizing composition into a coating composition into a suitable form for application as a spray, a dip, a paint, or a dilutable concentrate. The method may further comprise packaging the deodorizing composition, for instance in an aerosol spray can, a pump spray, or a bottle.

A dried form of the composition may be prepared by drying the liquid form to remove the diluent.

### Method of Applying the Deodorizing Composition to the Article

The present invention also provides a method of forming an absorbent article of the first aspect of the invention, the method comprising (a) coating or impregnating the article with the deodorizing composition; and (b) optionally drying the article.

In a particular embodiment, step (a) comprises impregnating the deodorizing composition within at least a part of the article. Step (a) may involve applying a quantity of the deodorizing composition to an absorbent/retaining layer or zone of an article (e.g. the absorbent layer of a sanitary towel), and may additionally or alternatively involve applying a quantity of the deodorizing composition to an acquisition layer or zone of an article (e.g. the acquisition layer of a sanitary towel). In some embodiments, step (a) is performed upon respective components of a pre-assembled absorbent article. For instance, for a sanitary towel, the deodorizing composition may be applied to the absorbent/retaining layer or zone and/or the acquisition layer prior to the assembly of the sanitary towel. For efficiency, the deodorizing composition is preferably applied to a continuous sheet of the absorbent/retaining layer or zone or a continuous sheet of an acquisition layer prior to said sheets being cut and subsequently assembled into the absorbent article (e.g. sanitary towel).

The deodorizing composition may be applied by spraying, pouring, squeezing, brushing, or wiping it onto or into the article, or alternatively by dipping the article or a part thereof into the deodorizing composition.

In a particular embodiment, coating and/or impregnating the article with a deodorizing composition involves coating and/or impregnating with a concentrate, suitably a concentrate spray. Most suitably, in this embodiment, the concentrate is applied to the article as a spray, preferably via a precision volumetric dosing unit. Precision volumetric dosing units suitably allow a deodorizing concentrate to be accurately and uniformly applied in minimal metered quantities across a wide area. This ensures good distribution of the concentrate and avoids the need for a comprehensive drying step, thus avoiding cumbersome processing steps and unnecessary energy consumption.

In accordance with an aspect of the present invention, there is provide a method of preparing a sanitary item (e.g. a sanitary towel), the method comprising (a) coating or impregnating the sanitary item with the concentrate; and (b) optionally drying the article. In a particular embodiment, the method does not involve drying the sanitary item. Suitably, coating or impregnating of the sanitary item involves spraying concentrate upon the sanitary item as defined herein.

An aspect of the invention provides a sanitary item (e.g. a sanitary towel) obtainable by, obtained by, or directly obtained by the method of preparing a sanitary item as defined herein.

An aspect of the invention provides a sanitary item (e.g. a sanitary towel) coated or impregnated with a concentrate as defined herein.

A further aspect of the invention provides a use of a concentrate as defined herein for coating or impregnating a sanitary item (e.g. a sanitary towel).

### EXAMPLES

### Materials

Polyhexamethylene biguanide, which was employed as an antimicrobial agent, was obtained commercially as Cosmocil CQ (20% strength), which is a 20% w/w aqueous solution obtainable from Arch Chemicals.

Methyl crotonate, which was employed as a deodorant, was commercially obtained as Sinodur from Givaudan.

Phospholipid, which was employed as an anti-irritant, was commercially obtained as Colalipid C from Colonial Chemicals Inc.

A preservative was commercially available as Nipaguard PO-05 available from Clariant.

### Example 1

### Deodorizing Mixture (Spray)

A deodorizing mixture was prepared by admixing all of the following ingredients (% values are weight percent of the total weight of the composition):

| | |
|---|---|
| 0.30% | Cosmocil CQ (20% strength) |
| 0.15% | Sinodur |
| 2.00% | Colalipid C |
| 0.80% | Nipaguard PO-05 |
| 0.50% | Lactic Acid |
| trace | Sodium Hydroxide (sufficient to adjust pH to be 4.0) |
| 96.25% | Ethanol |

The mixture, which was a clear alcoholic solution, was packaged into a spray for later application to a sanitary towel.

In an alternative embodiment, the deodorizing composition has less diluent (ethanol) but the same relative amounts of the other components, effectively providing a concentrate. The concentrate may be 5 to 25 times more concentrated (i.e. with 5 to 25 times less diluent). Although such concentrated deodorizing compositions must be applied to an article with a greater degree of accuracy, the overall process efficiency can improved by using a more concentrated composition (i.e. handling lower volumes, reduced drying times etc.).

### Example 2

### Deodorizing Mixture (Dip)

A deodorizing mixture was prepared by admixing all of the following ingredients:

| | |
|---|---|
| 3 g | Cosmocil CQ (20% strength) |
| 1.5 g | Sinodur |
| 20 g | Colalipid C |
| 8 g | Nipaguard PO-05 |
| 5 g | Lactic Acid |
| trace | Sodium Hydroxide (sufficient to adjust pH to be 4.0) |
| 962.5 g | Deionised water |

The mixture, which was a clear aqueous solution, was packaged into a dip for later application to a sanitary towel.

Again, in alternative embodiments, the deodorizing composition has less diluent (water) present but the same relative amounts of the other listed components, effectively providing a concentrate. The composition may be, for example, 10 to 20 times more concentrated (i.e. it comprises 10 or 20 times less diluent).

### Example 3

### Preparation of Sanitary Towel

A sanitary towel is prepared as a layered structure, with:

Layer 1—a comfort layer consisting of a non-absorbent perforated film (or woven synthetic material), which separates the user from the lower layers, and allows air circulation.
Layer 2—an acquisition layer consisting of an absorbent paper which holds the lower layers in place.
Layer 3—an absorbent core/layer consist of a superabsorbent polymer and cellulosic fibres arranged to received and hold secreted bodily fluids.
Layer 4—a non-absorbent film which sits below the absorbent layer to contain the bodily fluids.
Layer 5—a release paper which is removable from the bottom of the non-absorbent film to expose adhesive which adheres the sanitary towel inside an item of underwear.

A certain amount of the spray (based on the weight of the sanitary towel) of Example 1 is sprayed onto the underside of the acquisition layer (layer 2) and also onto the absorbent core (layer 3). In this example, the spray is applied to continuous sheets of each of the acquisition layer and absorbent core, before said sheets are cut to a suitable size and subsequently assembled into the multi-layered sanitary towel described above. The sanitary towel is then air dried to leave a final sanitary towel article composed of (% values are a weight % based on the overall weight of the sanitary towel):

| | |
|---|---|
| 0.30% | Cosmocil CQ (20% strength - hence this dries down to |
| | 0.06%) |
| 0.15% | Sinodur |
| 2.00% | Colalipid C |
| 0.80% | Nipaguard PO-05 |
| 0.50% | Lactic Acid |
| trace | Sodium Hydroxide |

In this example, the above quantities were obtained by impregnating the sanitary towel with 3.51 wt % pre-dried deodorizing composition, based on the total weight of the sanitary towel.

A sanitary towel having the same composition (in terms of deodorizing ingredients) and substantially the same structure as above, was produced by dipping the sanitary towel, comfort layer first, into a pre-measured amount of the dip of Example 2. Once the sanitary towel had absorbed all of the dip, it was allowed to air dry as described above. This demonstrates that deodorizing compositions may be provided to consumers as distinct products for selective application to absorbent articles such as sanitary towels before use.

### Example 4

### Model Studies: Testing of the Deodorizing Composition-Impregnated Sanitary Towels

### General Protocols

Experiments were conducted to assess the antimicrobial and deodorizing efficacy of treating three types of feminine hygiene pads (Lil-lets™)—Normal, Super and Night—with the GX deodorizing composition (a 20% w/v ethanolic solution of the undiluted form of the deodorizing composition of Example 1) against bacterial and yeast contaminants inoculated into a synthetic menstrual fluid matrix and this mix inoculated onto the pads. Two replicate tests were carried out on each type of pad over a time period of 0, 4 and 8 hours. Mixed bacterial inoculum and yeast inoculum was applied to two separate sets of treated and control pads. Bacterial species were later identified and counted using selective medium.

### Growth Media and Test Organisms

The growth media employed included:
Cysteine Lactose Electrolyte Deficient (CLED) agar
SLanetz Bartlay (SB) Medium
Malt Extract Ager (MEA)

The test organisms included:
Escherichia coli ATCC 10536
Proteus mirabilis NCTC 10374
Enterococcus faecalis NCTC 10927
Candida albicans ATCC 10231

### Preparation of Feminine Hygiene Pads

The test pads were the standard “Normal”, “Super” and “Night” pads available from Lil-lets at the filing date. Pads were treated with a total dose of 600 parts per million (ppm) of the GX deodorizing composition.

### Experimental Controls

Treated pads were compared with a matching control pad set over the same time points.

### Preparation of the GX Deodorizing Composition

The GX deodorizing composition was supplied as a 20% W/V solution in absolute ethanol (i.e. a concentrated form of the composition of Example 1, wherein the undiluted components comprise 20% w/v in ethanol).

### Contact Times

The pads were tested at 0, 4 and 8 hour intervals.

### Contact Temperature

The bacterial tests were conducted at 35+1° C.

### Organic Load

To simulate bodily fluid secretetion, a synthetic blood composition was used comprising: Sheep erythrocytes, 45% V/V; Foetal bovine serum, 50% V/V, 5 mM glucose and 0.25% W/V mucin.

### Pad Inoculation

Pads were inoculated with approximately 1×104 cfu/ml of each microbial strain in synthetic menstrual fluid in the following quantities:

TABLE 1

| | Amount of synthetic menstrual fluid |
|---|---|
| Towel type | added |
| Normal | 2.0 g |
| Super | 2.5 g |
| Night | 4.5 g |

### Test Agent Application

A 0.3 ml volume of a 20% W/V solution of the GX deodorizing composition was diluted in 4.7 ml of ethanol and applied evenly into the silica pulp of the pad.

### Neutralizer

The neutralizer employed comprised Lechithin, 0.3% W/V, Polysorbate 80, 2.0% W/V.

Test Equipment Used (with Calibration Details, as Applicable)
Haemocytometer
Incubator set at 30° C. GU-EQU-002, calibrated
Incubator set at 35° C., BT-EQU-124, calibrated
Incubator set at 37° C. BT-EQU-119, calibrated
OSPREY 50 L Autoclave (LTE) BT-EQU-117, calibrated
Class II Laminar Flow cabinet GU-EQU-004 (6 month external calibration cycle)
Spectrophotometer BT-EQU-154
Vacuum pump, BT-EQU-007; BT-EQU-073
Refrigerated centrifuge (Hiraeus) GU-EQU-009
Micropipettes, Regular service and calibration (6 month cycle).

Disposable sterile plasticware (Greiner, Sarstedt, Axygen).

### Reagents

The reagents employed are shown in Table 2:

TABLE 2

| Reagent | Lot Numbers | Source |
|---|---|---|
| Cysteine Lactose | BT-CMR-1269 | E & O laboratories, |
| electrolyte deficient | | Scotland, UK |
| (CLED) agar | | |
| SLanetz Bartlay (SB) | BT-2CMR-P0888 | Oxoid, UK |
| Medium | | |
| Malt Extract Ager (MEA) | BT-2CMR-P0838 | Oxoid, UK |
| TSA | BT-2CMR-P0881 | Oxoid, UK |
| Foetal bovine serum | BT-CMR-1147/1307 | Lonza |
| Sheep red blood cells | BT-CMR-1301/1302 | E & O laboratories, |
| | | Scotland, UK |
| Glucose | BT-CMR-039 | Sigma |
| Porcine mucin | BT-CMR-1290 | Sigma |
| Polysorbate 80 | BT-CMR-1125 | Acros |
| Lecithin | BT-CMR-1039 | Acros |

### Calculations

Total colony forming units recovered in 200 mls of neutralizer was calculated by plate count×dilution factor×2.

### Details of Procedure

Preparation of inocula was as follows (Table 3):

TABLE 3

| Bacteria/ | | | |
|---|---|---|---|
| Yeast | Day 1 | Day 2 | Day 3 |
| E. coli | culture TSA | culture TSA | culture synthetic blood |
| | | | without sheep red blood cells |
| | | | added (10 ml liquid culture |
| | | | at 37° C.) |
| P. mirabilis | culture TSA | culture TSA | culture synthetic blood |
| | | | without sheep red blood cells |
| | | | added (10 ml liquid culture at |
| | | | 37° C.) |
| E. faecalis | culture TSA | culture TSA | culture synthetic blood |
| | | | without sheep red blood cells |
| | | | added (10 ml liquid culture at |
| | | | 37° C.) |
| C. albicans | culture TSA | culture TSA | culture synthetic blood |
| | | | without sheep red blood cells |
| | | | added (10 ml liquid culture at |
| | | | 37° C.) |

### Bacteria

Monitor growth by absorbance at 620 nm to an estimate of 2.5×10(8) cfu/ml. Adjust by dilution in synthetic blood without sheep red blood cells to 1×10(6) cfu/ml.

Mix bacterial 1.0 ml of each bacterial strain add 47 mls of FBS and 5.0 mls of 100 mM glucose+5% mucin and add 45% V/V sheep erythrocytes to a total volume of 100.0 mls.

### Yeast

Count cells in a haemocytometer. Adjust by dilution in synthetic blood without sheep red blood cells to 1×10(6) cfu/ml mls.

Add 1.0 ml Candida+49 mls FBS+5.0 mls 100 mM Glucose+5% mucin add 45% V/V sheep erythrocytes to a total volume of 100.0.

### Synthetic Blood Composition

sheep red blood cells 45% v/v
Foetal bovine serum 50% v/v
100 mM glucose+5% mucin 5% v/v

### Treatment of Each Individual Pad

Inocula: Combined bacterial culture each bacterium at 1×10(4) cfu/ml or C. albicans at 1×10(4) cfu/ml

Inoculation was conducted so that “Normal”, Super”, and “Night” pads were each challenged by impregnating them respectively with 2.0 ml, 2.5 ml, and 4.5 ml of synthetic blood.

Each was incubated for a particular contact time, before adding 200 ml of neutralizer in sterile 250 ml/ or 500 ml bottles, and washing for 1 minute.

### Results

### Experiment 1 (Normal Pad)

Pads were either treated or untreated with 600 ppm of the GX deodorizing composition and this was allowed to dry. Two independent analyses were carried out for pooled bacterial inocula (E. coli, P. mirabilis, E. faecalis) and yeast inoculum (C. albicans). These were applied to the pad in a synthetic menstrual fluid matrix in a volume consistent with the mean weight of menstrual fluid determined previously in actual use of the pads. Pads were then incubated at 35° C.±1° C. for 0, 4 and 8 hours post inoculation in sterile glass bottles. Bacteria or yeast were recovered by gently washing for 1 minute in 200 mls neutralizer (0.3% W/V Lecithin, 2.0% W/V Polysorbate 80). Bacteria or yeast were then serially diluted from the neutralized mix. Bacteria were collected on filters and incubated in replicates on either CLED (selects E. coli, yellow colonies; P. mirabilis, blue colonies) or SB (E. faecalis) at 37° C.±1° C. Yeast were collected on filters and incubated on MEA at 30° C.±1° C. Counts in total colony forming units were calculated by plate count×2×dilution factor. Table 4 details the results:

TABLE 4

| | Untreated | | Treated | | Untreated | |
|---|---|---|---|---|---|---|
| | Pad Recovery | | Pad Recovery | | Pad Recovery | |
| Microbial | at 0 hrs. | | at 0 hrs. | | at 4 hrs. | |
| strain | (cfu) | mean | (cfu) | mean | (cfu) | mean |
| Escherichia | 3.40E+04 | | 4.80E+04 | | 3.40E+06 | |
| coli | 3.20E+04 | 3.30E+04 | 2.80E+04 | 3.80E+04 | 2.40E+06 | 2.90E+06 |
| Proteus | 3.00E+04 | | 4.00E+04 | | 6.00E+05 | |
| mirabilis | 4.60E+04 | 3.80E+04 | 3.60E+04 | 3.80E+04 | 1.40E+06 | 1.00E+06 |
| Enterococcus | 2.40E+04 | | 1.60E+04 | | 4.60E+06 | |
| faecalis | 2.20E+04 | 2.30E+04 | 2.20E+04 | 1.90E+04 | 5.40E+06 | 5.00E+06 |
| Candida | 7.40E+04 | | 6.20E+04 | | 7.30E+04 | |
| albicans | 7.60E+04 | 7.50E+04 | 7.40E+04 | 6.80E+04 | 9.20E+04 | 8.25E+04 |
| | Treated | | Untreated | | Treated | |
| | Pad Recovery | | Pad Recovery | | Pad Recovery | |
| Microbial | at 4 hrs. | | at 8 hrs. | | at 8 hrs. | |
| strain | (cfu) | mean | (cfu) | mean | (cfu) | mean |
| Escherichia | 2.48E+05 | | | | | |
| coli | 2.62E+05 | 2.55E+05 | | | | |
| Proteus | 1.32E+05 | | | | | |
| mirabilis | 1.28E+05 | 1.30E+05 | | | | |
| Enterococcus | 3.48E+05 | | | | | |
| faecalis | 2.70E+05 | 3.09E+05 | | | | |
| Candida | 2.00E+04 | | 8.48E+07 | | 4.86E+05 | |
| albicans | 2.40E+04 | 2.20E+04 | 8.36E+07 | 8.42E+07 | 3.34E+05 | 4.10E+05 |

The 8 hour time point experiment was repeated for bacteria. Pads were either treated or untreated with 600 ppm of PHMB and this was allowed to dry. Analyses were carried out for pooled bacterial inocula (E. coli, P. mirabilis, E. faecalis). These were applied to the pad in a synthetic menstrual fluid matrix in a volume consistent with the mean weight of menstrual fluid determined previously in actual use of the pads. Pads were then incubated at 35° C.±1° C. for 0 and 8 hours post inoculation in sterile glass bottles. Bacteria or yeast were recovered by gently washing for 1 minute in 200 mls neutralizer (0.3% W/V Lecithin, 2.0% W/V Polysorbate 80). Bacteria were then serially diluted from the neutralized mix. Bacteria were collected on filters and incubated in replicates on either CLED (selects E. coli, yellow colonies; P. mirabilis, blue colonies) or SB (E. faecalis) at 37° C.±1° C. Counts in total colony forming units were calculated by plate×count 2×dilution factor. Table 5 shows the results:

TABLE 5

| | Untreated | | Treated | | Untreated | | Treated | |
|---|---|---|---|---|---|---|---|---|
| | Pad | | Pad | | Pad | | Pad | |
| | Recovery at | | Recovery at | | Recovery at | | Recovery at | |
| Microbial | 0 hours. | | 0 hours. | | 8 hours. | | 8 hours. | |
| strain | (cfu) | mean | (cfu) | mean | (cfu) | mean | (cfu) | mean |
| Escherichia | 1.88E+04 | | 9.40E+03 | | 4.00E+08 | | 2.00E+06 | |
| coli | 1.64E+04 | 1.76E+04 | 1.40E+04 | 1.17E+04 | 4.80E+08 | 4.40E+08 | 2.00E+06 | 2.00E+06 |
| Proteus | 2.02E+04 | | 7.40E+03 | | 3.40E+09 | | 1.00E+07 | |
| mirabilis | 1.68E+04 | 1.85E+04 | 1.10E+04 | 9.20E+03 | 5.32E+09 | 4.36E+09 | 1.80E+07 | 1.40E+07 |
| Enterococcus | 1.58E+04 | | 1.02E+04 | | 1.70E+08 | | 2.00E+06 | |
| faecalis | 2.42E+04 | 2.00E+04 | 8.80E+03 | 9.50E+03 | 2.08E+08 | 1.89E+08 | 2.00E+06 | 2.00E+06 |

### Experiment 1 (Super Pad)

Pads were either treated or untreated with 600 ppm of the GX deodorizing composition and this was allowed to dry. Two independent analyses were carried out for pooled bacterial inocula (E. coli, P. mirabilis, E. faecalis) and yeast inoculum (C. albicans). These were applied to the pad in a synthetic menstrual fluid matrix in a volume consistent with the mean weight of menstrual fluid determined previously in actual use of the pads. Pads were then incubated at 35° C.±1° C. for 0, 4 and 8 hours post inoculation in sterile glass bottles. Bacteria or yeast were recovered by gently washing for 1 minute in 200 mls neutralizer (0.3% W/V Lecithin, 2.0% W/V Polysorbate 80). Bacteria or yeast were then serially diluted from the neutralized mix. Bacteria were collected on filters and incubated in replicates on either CLED (selects E. coli, yellow colonies; P. mirabilis, blue colonies) or SB (E. faecalis) at 37° C.±1° C. Yeast were collected on filters and incubated on MEA at 30° C.±1° C. Counts in total colony forming units were calculated by plate count×2×dilution factor. Table 6 shows the results:

TABLE 6

| | Untreated | | Treated | | Untreated | |
|---|---|---|---|---|---|---|
| | Pad Recovery | | Pad Recovery | | Pad Recovery | |
| Microbial | at 0 hours. | | at 0 hours. | | at 4 hours. | |
| strain | (cfu/ml) | | (cfu/ml) | | (cfu) | mean |
| Escherichia | 4.60E+04 | | 1.60E+01 | | 2.46E+07 | |
| coli | 6.60E+04 | 5.60E+04 | 2.00E+04 | 1.00E+04 | 2.10E+07 | 2.28E+07 |
| Proteus | 6.40E+04 | | 1.20E+04 | | 7.80E+06 | |
| mirabilis | 2.20E+04 | 4.30E+04 | 1.60E+04 | 1.40E+04 | 7.20E+06 | 7.50E+06 |
| Enterococcus | 2.00E+04 | | 2.60E+04 | | 2.70E+07 | |
| faecalis | 1.80E+04 | 1.90E+04 | 6.60E+04 | 4.60E+04 | 2.46E+07 | 2.58E+07 |
| Candida | 7.20E+04 | | 1.12E+05 | | 3.40E+05 | |
| albicans | 5.40E+04 | 6.30E+04 | 1.26E+05 | 1.19E+05 | 3.50E+05 | 3.45E+05 |
| | Treated | | Untreated | | Treated | |
| | Pad Recovery | | Pad Recovery | | Pad Recovery | |
| Microbial | at 4 hours. | | at 8 hours. | | at 8 hours. | |
| strain | (cfu) | mean | (cfu) | mean | (cfu) | mean |
| Escherichia | 8.00E+05 | | | | | |
| coli | 6.00E+05 | 7.00E+05 | | | | |
| Proteus | 2.00E+05 | | | | | |
| mirabilis | 1.00E+05 | 1.50E+05 | | | | |
| Enterococcus | 8.12E+05 | | | | | |
| faecalis | 6.24E+05 | 7.18E+05 | | | | |
| Candida | 1.60E+04 | | 1.80E+06 | | 2.32E+05 | |
| albicans | 2.60E+04 | 2.10E+04 | 4.20E+06 | 3.00E+06 | 2.48E+05 | 2.40E+05 |

The 8 hour time point experiment was repeated for bacteria. Pads were either treated or untreated with 600 ppm of PHMB and this was allowed to dry. Analyses were carried out for pooled bacterial inocula (E. coli, P. mirabilis, E. faecalis). These were applied to the pad in a synthetic menstrual fluid matrix in a volume consistent with the mean weight of menstrual fluid determined previously in actual use of the pads. Pads were then incubated at 35° C.±1° C. for 0 and 8 hours post inoculation in sterile glass bottles. Bacteria or yeast were recovered by gently washing for 1 minute in 200 mls neutralizer (0.3% W/V Lecithin, 2.0% W/V Polysorbate 80). Bacteria were then serially diluted from the neutralized mix. Bacteria were collected on filters and incubated in replicates on either CLED (selects E. coli, yellow colonies; P. mirabilis, blue colonies) or SB (E. faecalis) at 37° C.±1° C. Counts in total colony forming units were calculated by plate count×2×dilution factor. Table 7 shows the results:

TABLE 7

| | Untreated | | Treated | | Untreated | | Treated | |
|---|---|---|---|---|---|---|---|---|
| | Pad | | Pad | | Pad | | Pad | |
| | Recovery | | Recovery | | Recovery | | Recovery | |
| Microbial | at 0 hours. | | at 0 hours. | | at 8 hours. | | at 8 hours. | |
| strain | (cfu) | mean | (cfu) | mean | (cfu) | mean | (cfu) | mean |
| Escherichia | 3.72E+04 | | 2.70E+04 | | 3.28E+09 | | 2.62E+08 | |
| coli | 3.94E+04 | 3.83E+04 | 2.80E+04 | 2.75E+04 | 2.76E+09 | 3.02E+09 | 2.52E+08 | 2.57E+08 |
| Proteus | 3.52E+04 | | 2.08E+04 | | 3.88E+09 | | 3.42E+08 | |
| mirabilis | 2.62E+04 | 3.07E+04 | 1.78E+04 | 1.93E+04 | 2.08E+09 | 2.98E+09 | 3.64E+08 | 3.53E+08 |
| Enterococcus | 4.00E+04 | | 2.58E+04 | | 8.40E+08 | | 1.28E+08 | |
| faecalis | 3.66E+04 | 3.83E+04 | 2.78E+04 | 2.68E+04 | 9.20E+08 | 8.80E+08 | 1.68E+08 | 1.48E+08 |

### Experiment 1 (Night Pad)

Pads were either treated or untreated with 600 ppm of the GX deodorizing composition and this was allowed to dry. Two independent analyses were carried out for pooled bacterial inocula (E. coli, P. mirabilis, E. faecalis) and yeast inoculum (C. albicans). These were applied to the pad in a synthetic menstrual fluid matrix in a volume consistent with the mean weight of menstrual fluid determined previously in actual use of the pads. Pads were then incubated at 35° C.±1° C. for 0, 4 and 8 hours post inoculation in sterile glass bottles. Bacteria or yeast were recovered by gently washing for 1 minute in 200 mls neutralizer (0.3% W/V Lecithin, 2.0% W/V Polysorbate 80). Bacteria or yeast were then serially diluted from the neutralized mix. Bacteria were collected on filters and incubated in replicates on either CLED (selects E. coli, yellow colonies; P. mirabilis, blue colonies) or SB (E. faecalis) at 37° C.±1° C. Yeast were collected on filters and incubated on MEA at 30° C.±1° C. Counts in total colony forming units were calculated by plate count×2×dilution factor. Table 8 shows the results:

TABLE 8

| | Untreated | | Treated | | Untreated | |
|---|---|---|---|---|---|---|
| | Pad Recovery | | Pad Recovery | | Pad Recovery | |
| Microbial | at 0 hours. | | at 0 hours. | | at 4 hours. | |
| strain | (cfu) | mean | (cfu) | mean | (cfu) | mean |
| Escherichia | 1.30E+05 | | 2.00E+03 | | 4.84E+07 | |
| coli | 1.26E+05 | 1.28E+05 | 2.00E+03 | 2.00E+03 | 4.54E+07 | 4.69E+07 |
| Proteus | 8.40E+04 | | 1.14E+05 | | 1.34E+07 | |
| mirabilis | 1.18E+05 | 1.01E+05 | 1.12E+05 | 1.13E+05 | 1.18E+07 | 1.26E+07 |
| Enterococcus | 1.44E+05 | | 2.00E+03 | | 5.24E+07 | |
| faecalis | 1.08E+05 | 1.26E+05 | 2.00E+03 | 2.00E+03 | 5.46E+07 | 5.35E+07 |
| Candida | 1.64E+05 | | 8.20E+04 | | 4.94E+05 | |
| albicans | 1.42E+05 | 1.53E+05 | 9.40E+04 | 8.80E+04 | 3.70E+05 | 4.32E+05 |
| | Treated | | Untreated | | Treated | |
| | Pad Recovery | | Pad Recovery | | Pad Recovery | |
| Microbial | at 4 hours. | | at 8 hours. | | at 8 hours. | |
| strain | (cfu) | mean | (cfu) | mean | (cfu) | mean |
| Escherichia | 5.20E+06 | | | | | |
| coli | 2.20E+06 | 3.70E+06 | | | | |
| Proteus | 1.80E+06 | | | | | |
| mirabilis | 2.20E+06 | 2.00E+06 | | | | |
| Enterococcus | 7.12E+05 | | | | | |
| faecalis | 5.64E+05 | 6.38E+05 | | | | |
| Candida | 5.20E+04 | | 2.84E+07 | | 3.16E+05 | |
| albicans | 4.60E+04 | 4.90E+04 | 2.42E+07 | 2.63E+07 | 3.02E+05 | 3.09E+05 |

The 8 hour time point experiment was repeated for bacteria. Pads were either treated or untreated with 600 ppm of PHMB and this was allowed to dry. Analyses were carried out for pooled bacterial inocula (E. coli, P. mirabilis, E. faecalis). These were applied to the pad in a synthetic menstrual fluid matrix in a volume consistent with the mean weight of menstrual fluid determined previously in actual use of the pads. Pads were then incubated at 35° C.±1° C. for 0 and 8 hours post inoculation in sterile glass bottles. Bacteria or yeast were recovered by gently washing for 1 minute in 200 mls neutralizer (0.3% W/V Lecithin, 2.0% W/V Polysorbate 80). Bacteria were then serially diluted from the neutralized mix. Bacteria were collected on filters and incubated in replicates on either CLED (selects E. coli, yellow colonies; P. mirabilis, blue colonies) or SB (E. faecalis) at 37° C.±1° C. Counts in total colony forming units were calculated by plate count×2×dilution factor. Table 9 shows the results.

TABLE 9

| | Untreated | | Treated | | Untreated | | Treated | |
|---|---|---|---|---|---|---|---|---|
| | Pad | | Pad | | Pad | | Pad | |
| | Recovery | | Recovery | | Recovery | | Recovery | |
| Microbial | at 0 hours. | | at 0 hours. | | at 8 hours. | | at 8 hours. | |
| strain | (cfu) | mean | (cfu) | mean | (cfu) | mean | (cfu) | mean |
| Escherichia | 9.20E+04 | | 7.40E+04 | | 1.58E+09 | | 2.00E+06 | |
| coli | 9.00E+04 | 9.10E+04 | 9.00E+04 | 8.20E+04 | 1.76E+09 | 1.67E+09 | 2.00E+06 | 2.00E+06 |
| Proteus | 7.20E+04 | | 7.60E+04 | | 8.10E+09 | | 2.00E+07 | |
| mirabilis | 6.40E+04 | 6.80E+04 | 1.22E+05 | 9.90E+04 | 7.88E+09 | 7.99E+09 | 3.00E+07 | 2.50E+07 |
| Enterococcus | 7.20E+04 | | 9.40E+04 | | 1.36E+09 | | 8.00E+06 | |
| faecalis | 6.20E+04 | 6.70E+04 | 7.20E+04 | 8.30E+04 | 1.24E+09 | 1.30E+09 | 2.00E+06 | 5.00E+06 |

### Experiment 2 (Normal Pad)

Pads were either treated or untreated with 600 ppm of the GX deodorizing composition and this was allowed to dry. Two independent analyses were carried out for pooled bacterial inocula (E. coli, P. mirabilis, E. faecalis) and yeast inoculum (C. albicans). These were applied to the pad in a synthetic menstrual fluid matrix in a volume consistent with the mean weight of menstrual fluid determined previously in actual use of the pads. Pads were then incubated at 35° C.±1° C. for 0, 4 and 8 hours post inoculation in sterile glass bottles. Bacteria or yeast were recovered by gently washing for 1 minute in 200 mls neutralizer (0.3% W/V Lecithin, 2.0% W/V Polysorbate 80). Bacteria or yeast were then serially diluted from the neutralized mix. Bacteria were collected on filters and incubated in replicates on either CLED (selects E. coli, yellow colonies; P. mirabilis, blue colonies) or SB (E. faecalis) at 37° C.±1° C. Yeast were collected on filters and incubated on MEA at 30° C.±1° C. Counts in total colony forming units were calculated by plate count×2×dilution factor. Table 10 shows the results:

TABLE 10

| | Untreated | | Treated | | Untreated | |
|---|---|---|---|---|---|---|
| | Pad Recovery | | Pad Recovery | | Pad Recovery | |
| Microbial | at 0 hours. | | at 0 hours. | | at 4 hours. | |
| strain | (cfu) | mean | (cfu) | mean | (cfu) | mean |
| Escherichia | 4.00E+03 | | 6.00E+03 | | 3.40E+04 | |
| coli | 4.00E+03 | 4.00E+03 | 1.60E+04 | 1.10E+04 | 3.60E+04 | 3.50E+04 |
| Proteus | 2.00E+03 | | 2.00E+03 | | 1.72E+05 | |
| mirabilis | 1.00E+03 | 1.50E+03 | 6.00E+03 | 4.00E+03 | 1.72E+05 | 1.72E+05 |
| Enterococcus | 1.00E+03 | | 2.00E+03 | | 1.78E+05 | |
| faecalis | 2.00E+03 | 1.50E+03 | 1.00E+03 | 1.50E+03 | 1.98E+05 | 1.88E+05 |
| Candida | 1.10E+03 | | 6.20E+02 | | 4.00E+03 | |
| albicans | 6.00E+02 | 8.50E+02 | 7.80E+02 | 7.00E+02 | 2.00E+03 | 3.00E+03 |
| | Treated | | Untreated | | Treated | |
| | Pad Recovery | | Pad Recovery | | Pad Recovery | |
| Microbial | at 4 hours. | | at 8 hours. | | at 8 hours. | |
| strain | (cfu) | mean | (cfu) | mean | (cfu) | mean |
| Escherichia | 4.00E+03 | | 7.20E+08 | | 6.40E+07 | |
| coli | 2.00E+03 | 3.00E+03 | 6.20E+08 | 6.70E+08 | 5.80E+07 | 6.10E+07 |
| Proteus | 2.00E+03 | | 1.18E+08 | | 8.00E+06 | |
| mirabilis | 2.00E+03 | 2.00E+03 | 1.40E+08 | 1.29E+08 | 1.20E+07 | 1.00E+07 |
| Enterococcus | 4.00E+03 | | 1.22E+08 | | 4.00E+06 | |
| faecalis | 2.00E+03 | 3.00E+03 | 1.26E+08 | 1.24E+08 | 8.00E+06 | 6.00E+06 |
| Candida | 2.00E+03 | | 6.00E+03 | | 2.00E+03 | |
| albicans | 4.00E+03 | 3.00E+03 | 4.00E+03 | 5.00E+03 | 2.00E+03 | 2.00E+03 |

### Experiment 2 (Super Pad)

Pads were either treated or untreated with 600 ppm of PHMB and this was allowed to dry. Two independent analyses were carried out for pooled bacterial inocula (E. coli, P. mirabilis, E. faecalis) and yeast inoculum (C. albicans). These were applied to the pad in a synthetic menstrual fluid matrix in a volume consistent with the mean weight of menstrual fluid determined previously in actual use of the pads. Pads were then incubated at 35° C.±1° C. for 0, 4 and 8 hours post inoculation in sterile glass bottles. Bacteria or yeast were recovered by gently washing for 1 minute in 200 mls neutralizer (0.3% W/V Lecithin, 2.0% W/V Polysorbate 80). Bacteria or yeast were then serially diluted from the neutralized mix. Bacteria were collected on filters and incubated in replicates on either CLED (selects E. coli, yellow colonies; P. mirabilis, blue colonies) or SB (E. faecalis) at 37° C.±1° C. Yeast were collected on filters and incubated on MEA at 30° C.±1° C. Counts in total colony forming units were calculated by plate count×2×dilution factor. Table 11 shows the results:

TABLE 11

| | Untreated | | Treated | | Untreated | |
|---|---|---|---|---|---|---|
| | Pad Recovery | | Pad Recovery | | Pad Recovery | |
| Microbial | at 0 hours. | | at 0 hours. | | at 4 hours. | |
| strain | (cfu) | mean | (cfu) | mean | (cfu) | mean |
| Escherichia | 4.00E+03 | | 2.00E+03 | | 8.00E+03 | |
| coli | 2.00E+03 | 3.00E+03 | 2.00E+03 | 2.00E+03 | 6.00E+03 | 7.00E+03 |
| Proteus | 2.00E+03 | | 2.00E+03 | | 4.96E+05 | |
| mirabilis | 2.00E+03 | 2.00E+03 | 2.00E+03 | 2.00E+03 | 5.06E+05 | 5.01E+05 |
| Enterococcus | 2.00E+03 | | 2.00E+03 | | 5.00E+05 | |
| faecalis | 2.00E+03 | 2.00E+03 | 2.00E+03 | 2.00E+03 | 4.82E+05 | 4.91E+05 |
| Candida | 1.70E+03 | | 1.62E+03 | | 2.00E+03 | |
| albicans | 1.54E+03 | 1.62E+03 | 1.72E+03 | 1.67E+03 | 2.00E+03 | 2.00E+03 |
| | Treated | | Untreated | | Treated | |
| | Pad Recovery | | Pad Recovery | | Pad Recovery | |
| Microbial | at 4 hours. | | at 8 hours. | | at 8 hours. | |
| strain | (cfu) | mean | (cfu) | mean | (cfu) | mean |
| Escherichia | 1.80E+04 | | 1.62E+08 | | 5.80E+07 | |
| coli | 8.00E+03 | 1.30E+04 | 1.64E+08 | 1.63E+08 | 5.00E+07 | 5.40E+07 |
| Proteus | 8.00E+03 | | 4.00E+07 | | 1.20E+07 | |
| mirabilis | 1.80E+04 | 1.30E+04 | 5.60E+07 | 4.80E+07 | 1.20E+07 | 1.20E+07 |
| Enterococcus | 2.40E+04 | | 5.20E+07 | | 1.40E+07 | |
| faecalis | 3.20E+04 | 2.80E+04 | 3.60E+07 | 4.40E+07 | 4.00E+06 | 9.00E+06 |
| Candida | 4.00E+03 | | 1.60E+04 | | 2.00E+03 | |
| albicans | 1.00E+04 | 7.00E+03 | 8.00E+03 | 1.20E+04 | 4.00E+03 | 3.00E+03 |

### Experiment 2 (Night Pad)

Pads were either treated or untreated with 600 ppm of the GX deodorizing composition and this was allowed to dry. Two independent analyses were carried out for pooled bacterial inocula (E. coli, P. mirabilis, E. faecalis) and yeast inoculum (C. albicans). These were applied to the pad in a synthetic menstrual fluid matrix in a volume consistent with the mean weight of menstrual fluid determined previously in actual use of the pads. Pads were then incubated at 35° C.±1° C. for 0, 4 and 8 hours post inoculation in sterile glass bottles. Bacteria or yeast were recovered by gently washing for 1 minute in 200 mls neutralizer (0.3% W/V Lecithin, 2.0% W/V Polysorbate 80). Bacteria or yeast were then serially diluted from the neutralized mix. Bacteria were collected on filters and incubated in replicates on either CLED (selects E. coli, yellow colonies; P. mirabilis, blue colonies) or SB (E. faecalis) at 37° C.±1° C. Yeast were collected on filters and incubated on MEA at 30° C.±1° C. Counts in total colony forming units were calculated by plate count×2×dilution factor. The results are shown in Table 12:

TABLE 12

| | Untreated | | Treated | | Untreated | |
|---|---|---|---|---|---|---|
| | Pad Recovery | | Pad Recovery | | Pad Recovery | |
| Microbial | at 0 hours. | | at 0 hours. | | at 4 hours. | |
| strain | (cfu) | mean | (cfu) | mean | (cfu) | mean |
| Escherichia | 4.00E+04 | | 8.00E+03 | | 7.00E+04 | |
| coli | 2.60E+04 | 3.30E+04 | 8.00E+03 | 8.00E+03 | 8.60E+04 | 7.80E+04 |
| Proteus | 2.00E+04 | | 4.00E+03 | | 5.30E+05 | |
| mirabilis | 1.00E+04 | 1.50E+04 | 4.00E+03 | 4.00E+03 | 6.04E+05 | 5.67E+05 |
| Enterococcus | 1.40E+04 | | 8.00E+03 | | 5.52E+05 | |
| faecalis | 6.00E+03 | 1.00E+04 | 1.60E+04 | 1.20E+04 | 5.20E+05 | 5.36E+05 |
| Candida | 2.14E+03 | | 2.90E+03 | | 8.00Et03 | |
| albicans | 2.06E+03 | 2.10E+03 | 2.62E+03 | 2.76E+03 | 6.00E+03 | 7.00E+03 |
| | Treated | | Untreated | | Treated | |
| | Pad Recovery | | Pad Recovery | | Pad Recovery | |
| Microbial | at 4 hours. | | at 8 hours. | | at 8 hours. | |
| strain | (cfu) | mean | (cfu) | mean | (cfu) | mean |
| Escherichia | 8.00E+03 | | 5.78E+08 | | 4.00E+07 | |
| coli | 6.00E+03 | 7.00E+03 | 5.50E+08 | 5.64E+08 | 2.20E+07 | 3.10E+07 |
| Proteus | 2.00E+03 | | 3.14E+08 | | 2.00E+06 | |
| mirabilis | 2.00E+03 | 2.00E+03 | 2.56E+08 | 2.85E+08 | 2.00E+06 | 2.00E+06 |
| Enterococcus | 2.00E+03 | | 3.30E+08 | | 2.00E+06 | |
| faecalis | 2.00E+03 | 2.00E+03 | 3.44E+08 | 3.37E+08 | 2.00E+06 | 2.00E+06 |
| Candida | 2.00E+03 | | 5.00E+04 | | 2.00E+03 | |
| albicans | 2.00E+03 | 2.00E+03 | 3.40E+04 | 4.20E+04 | 2.00E+33 | 2.00E+03 |

### Summary of Microbial Growth Inhibition Data

Tables 13, 14, and 15 show microbial growth inhibition date (microorganisms recovered in untreated pads/microorganisms recovered in treated pads) for Normal, Super, and Night Pads respectively.

TABLE 13
Normal Pad type

| | EXPERIMENT 1 | EXPERIMENT 2 | EXPERIMENT 1 | EXPERIMENT 2 |
|---|---|---|---|---|
| | 4.0 hour mean | 4.0 hour mean | 8.0 hour mean | 8.0 hour mean |
| | reduction in | reduction in | reduction in | reduction in |
| Microbial strain | growth | growth | growth | growth |
| Escherichia coli | 1.14E+01 | 5.38E−01 | 2.20E+02 | 1.10E+01 |
| Proteus mirabilis | 7.69E+00 | 3.85E+01 | 3.11E+02 | 1.29E+01 |
| Enterococcus faecalis | 1.62E+01 | 1.75E+01 | 9.45E+01 | 2.07E+01 |
| Candida albicans | 3.75E+00 | 2.86E−01 | 2.05E+02 | 2.50E+00 |

TABLE 14
Super Pad type

| | EXPERIMENT 1 | EXPERIMENT 2 | EXPERIMENT 1 | EXPERIMENT 2 |
|---|---|---|---|---|
| | 4.0 hour mean | 4.0 hour mean | 8.0 hour mean | 8.0 hour mean |
| | reduction in | reduction in | reduction in | reduction in |
| Microbial strain | growth | growth | growth | growth |
| Escherichia coli | 3.26E+01 | 5.38E−01 | 1.18E+01 | 3.02E+00 |
| Proteus mirabilis | 5.00E+01 | 3.85E+01 | 8.44E+00 | 4.00E+00 |
| Enterococcus faecalis | 3.59E+01 | 1.75E+01 | 5.95E+00 | 4.89E+00 |
| Candida albicans | 1.64E+01 | 2.86E−01 | 1.25E+01 | 4.00E+00 |

TABLE 15
Night Pad type

| | EXPERIMENT 1 | EXPERIMENT 2 | EXPERIMENT 1 | EXPERIMENT 2 |
|---|---|---|---|---|
| | 4.0 hour mean | 4.0 hour mean | 8.0 hour mean | 8.0 hour mean |
| | reduction in | reduction in | reduction in | reduction in |
| Microbial strain | growth | growth | growth | growth |
| Escherichia coli | 1.27E+01 | 1.11E+01 | 8.35E+02 | 1.82E+01 |
| Proteus mirabilis | 6.30E+00 | 2.84E+02 | 3.20E+02 | 1.43E+02 |
| Enterococcus faecalis | 8.39E+01 | 2.68E+02 | 2.60E+02 | 1.69E+02 |
| Candida albicans | 8.82E+00 | 3.50E+00 | 8.51E+01 | 2.10E+01 |

### Odour Tests

Pads, “Normal”, “Super” and “Night” were either treated or untreated with 600 ppm of the GX deodorizing composition and this was allowed to dry. Pooled bacterial inocula (E. coli, P. mirabilis, E. faecalis) and yeast inoculum (C. albicans) were applied to the pad in a synthetic menstrual fluid matrix in a volume consistent with the mean weight of menstrual fluid determined previously in actual use of the pads. Pads were then incubated at 35° C.±1° C. for 0 and 6 hours post inoculation in sterile glass bottles. These were then randomly labelled from 1 to 12. These were then scored for odour on a scale of 1-5 independently by 6 volunteers. In the diagram mean scores for each pad either treated or untreated with the GX deodorizing composition, at 0 and 6 hours incubation are shown. Error bars are standard deviations.

Table 16 shows the data for scoring odour for different bacteria and yeast inoculated pads either treated or untreated with 600 ppm the GX deodorizing composition after incubation for 0 or 6 hours at 35° C.±1° C.

TABLE 16

| SAMPLE | MB | KF | SG | LM | PM | CW | Mean | St dev | |
|---|---|---|---|---|---|---|---|---|---|
| 1 | 1 | 3 | 1 | 3 | 3 | 1 | 2.00 | 1.095445 | Normal treated 6 hours |
| 2 | 5 | 4 | 4 | 5 | 5 | 5 | 4.67 | 0.516398 | Normal control 6 hours |
| 3 | 2 | 1 | 1 | 1 | 1 | 2 | 1.33 | 0.516398 | Night control 0 hours |
| 4 | 3 | 2 | 1 | 3 | 2 | 2 | 2.17 | 0.752773 | Super treated 6 hours |
| 5 | 3 | 3 | 1 | 1 | 4 | 1 | 2.17 | 1.32916 | Normal Treated 0 hours |
| 6 | 4 | 1 | 3 | 3 | 3 | 1 | 2.50 | 1.224745 | Normal control 0 hours |
| 7 | 1 | 2 | 1 | 3 | 2 | 1 | 1.67 | 0.816497 | Night treated 0 hours |
| 8 | 3 | 4 | 2 | 5 | 3 | 1 | 3.00 | 1.414214 | Night treated 6 hours |
| 9 | 4 | 1 | 4 | 5 | 1 | 5 | 3.33 | 1.861899 | Super control 6 hours |
| 10 | 1 | 2 | 4 | 2 | 2 | 3 | 2.33 | 1.032796 | Super control 0 hours |
| 11 | 5 | 3 | 4 | 5 | 3 | 3 | 3.83 | 0.983192 | Night control 6 hours |
| 12 | 2 | 3 | 1 | 3 | 4 | 2 | 2.50 | 1.048809 | Super treated 0 hours |
| | 2.83 | 2.42 | 2.25 | 3.25 | 2.75 | 2.25 | | | |
| | 1.47 | 1.08 | 1.42 | 1.48 | 1.22 | 1.48 | | | |

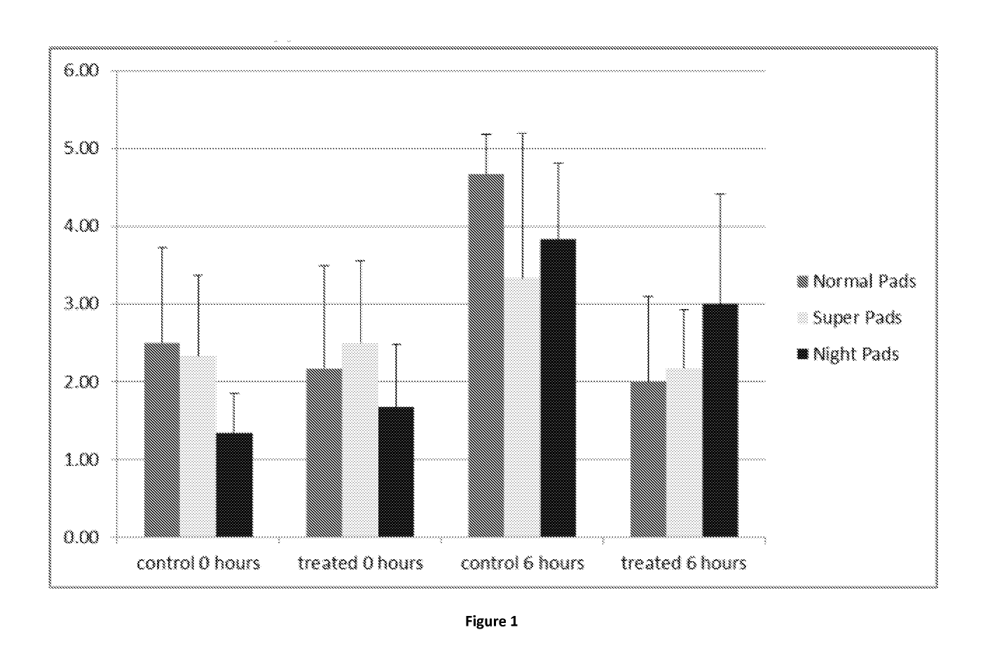

These results are summarized in FIG. 1.

### Discussion

The antimicrobial action of 600 ppm of the GX deodorizing composition on odour forming bacteria found in human excretions has been investigated on three types of Lil-lets pad; “Normal”, “Super”, and “Night”. Bacteria grew rapidly within the synthetic menstrual fluid matrix within the pad during the 8 hour period of analysis reacting levels up to 107-109 cfu from initial inocula of 103-105 cfu. Yeast growth was less rapid, achieving levels of 105 cfu from initial inocula of 103-105 cfu. There were in general no differences observed between the type of pad, although higher levels of growth were observed in Night pads that had received heavier inoculums and growth inhibition was slightly less in Super pads after 8 hours compared to the other types of pads. Treatment with 600 ppm of the GX deodorizing composition consistently resulted in reductions in growth in the range 101 to 102 at 4 hours and 8 hours after inoculation. Pads inoculated with a mixed bacterial and east inoculums scored the most highly for odour by 6 volunteers at 6 hours in untreated, but not treated pads, indicating the deodorizing effect of the GX deodorizing composition. Levels of odour in the treated pads at 6 hours were not distinguished from pads either treated or untreated that had not been incubated.

The results demonstrate that compositions of the present invention reduce odours, control pH to within non-irritable limits, and successfully restrict microbial growth. As such, the deodorizing compositions of the present invention provide enhanced comfort and reduced odours over extended use times.

### Example 5

### Sanitary Towel Impregnated with Deodorizing Concentrate (Spray)

### Preparation of Deodorizing Concentrates

Two separate deodorizing concentrate mixtures (Concentrate 1 and Concentrate 2) were prepared by admixing all of the following ingredients (% values are weight percent of the total weight of the composition):

CONCENTRATE 1

| Description | Quantity/1000 KG |
|---|---|
| Vantocil TG | 300 kg |
| Deionised water | 300 kg |
| Deoplex Clear H4699 | 300 kg |
| Colalipid C | 98 kg |
| Lactic Acid | 2 kg |
| TOTAL | 1000 kg |

CONCENTRATE 2

| Description | Quantity/1000 KG |
|---|---|
| Vantocil TG | 300 kg |
| Deoplex Clear H4699 | 600 kg |
| Colalipid C | 98 kg |
| Lactic Acid | 2 kg |
| TOTAL | 1000 kg |

Concentrates 1 and 2 were both clear liquids. Both were then packaged into a spray for later application to a sanitary towel.

Both Concentrates 1 and 2 were found to perform well in terms of their biocidal, deodorizing, and anti-irritancy properties, to provide good value and to enhance the effectiveness of biocides when used within the relevant safety limits.

Preparation of Impregnated Sanitary Towels with Concentrates 1 and 2

First and second sanitary towels were each prepared with a layered structure, as per Example 3 above.

To produce a first impregnated sanitary towel, 0.06 g of Concentrate 1 Spray was then uniformly dispensed/sprayed onto both the underside of the acquisition layer (layer 2) and also onto the absorbent core (layer 3) of the first sanitary towel, using a precision volumetric dosing unit, such as those available from Intertronic which are currently used in industry to accurately dispense adhesives, fragrances, dyes and the like. Due to the accurate and minimal dosing of the Concentrate 1 Spray, there was no need for an external drying step. In this example, the above quantities correspond to impregnating the first sanitary towel with 1.2 wt % deodorizing Concentrate 1, based on the total weight of the sanitary towel.

To produce a second impregnated sanitary towel, 0.06 g of Concentrate 2 Spray was uniformly dispensed/sprayed onto both the underside of the acquisition layer (layer 2) and also onto the absorbent core (layer 3) of the second sanitary towel, using the same precision volumetric dosing unit as used in the first example. Again, due to the accurate and minimal dosing of the Concentrate 2 Spray, there was no need for an external drying step. In this example, the above quantities correspond to impregnating the second sanitary towel with 1.2 wt % deodorizing Concentrate 2, based on the total weight of the sanitary towel.

Both the above impregnated sanitary towels were found to perform extremely well, provide excellent value, and allow for cheap and straightforward manufacturing.

1. An absorbent article configured to absorb and retain bodily fluids (e.g. menstrual fluids, urine, blood or sweat) during use, wherein the article is coated or impregnated with a deodorizing composition comprising a buffering component, a deodorizing agent, and an antimicrobial agent.

2. The absorbent article as claimed in claim 1, wherein the article is an article for use on, or in close proximity to, the human body.

3. The absorbent article as claimed in any preceding claim, wherein the article is selected from the group comprising sanitary items (e.g. sanitary towels, tampons), incontinence pads, nappies, sweatbands, items of clothing (e.g. underwear), footwear (e.g. the soles of shoes, slippers, boots, trainers etc.), headgear (e.g. hats or helmets); linen (e.g. bed linen); towels or medical fabrics (e.g. bandages, wound dressings).

4. The absorbent article as claimed in any preceding claim, wherein the article is suitably a hygiene product.

5. The absorbent article as claimed in claim 4, wherein the article is a sanitary towel.

6. The absorbent article as claimed in claim 1 or 2, wherein the article is an absorbent article such as sanitary towel, incontinence pad or nappy, which comprises an absorbent absorbent/retaining layer or zone, which receives and retains the bodily fluid within the article, and, optionally, a separate acquisition layer or zone through which the bodily fluids flow as they are absorbed into the absorbent/retaining layer or zone of the article, and wherein the deodorizing composition is present in the absorbent/retaining layer or zone and/or the acquisition layer or zone.

7. The absorbent article as claimed in any preceding claim, wherein the article comprises sufficient deodorizing composition to provide (based on the total weight of the article):
0.001 to 0.05 wt % of buffering component
0.05 to 0.2 wt % of antimicrobial agent
0.1 to 1 wt % of deodorizing agent.

8. The absorbent article as claimed in any preceding claim, wherein the deodorizing composition creates a localised acidic environment within the article when bodily fluids are absorbed into the article, such that the localised acidic environment has a pH of between 3.5 and 5.5.

9. A deodorizing composition comprising a buffering component, a deodorizing agent, and an antimicrobial agent.

10. The deodorizing composition as claimed in claim 9, wherein the deodorizing composition comprises the buffering component, deodorizing agent, and antimicrobial agent in a weight ratio of 1-3:100-1000:10-400 (buffering component:deodorizing agent:antimicrobial agent).

11. The deodorizing composition as claimed in claim 9, wherein the deodorizing composition comprises the buffering component, deodorizing agent, and antimicrobial agent in a weight ratio of 2:200-800:20-100 (buffering component:deodorizing agent:antimicrobial agent).

12. The deodorizing composition as claimed in any of claims 9 to 11, wherein the weight ratio of the buffering component to the antimicrobial agent is 10:60 to 1:300.

13. The deodorizing composition as claimed in any of claims 9 to 12, wherein the buffering component comprises an acid, wherein the acid has a pKₐ between 2 and 5.

14. The deodorizing composition as claimed in any of claims 9 to 13, wherein the antimicrobial agent is selected from the group including quaternary ammonium compounds (such as those well-known in the art, e.g. alkyldimethyl benzylammonium chloride), didecyl dimethyl ammonium chloride, amphoterics, chlorhexidine gluconate, polyhexamethylene biguanide (PHMB), or an antimicrobial metal-containing compound (e.g. titanium dioxide (TiO₂)), or a combination thereof.

15. The deodorizing composition as claimed in any of claims 9 to 14, wherein the composition further comprises a phospholipid component.

16. The deodorizing composition as claimed in any of claims 9 to 15, wherein the deodorizing agent comprises a deodorant compound selected from the group including methyl crotonate (e.g. Sinodur™ from Givaudan), Deoplex™ (obtainable from Chemlink Specialities), Deoplex™ (Saccharomyces ferment by Carrubba Inc.), sodium ricinoleate (obtainable from Chemlink Specialities), zinc ricinoleate (Flexisorb™ by ICT Inc.), cyclodextrin (obtainable from Proctor & Gamble), or a combination thereof.

17. A method of manufacturing a deodorizing composition (in liquid form) the method comprising mixing a buffering component, a deodorizing agent and an antimicrobial agent in a suitable diluent; and optionally adjusting the pH of the composition.

18. A method of forming an absorbent article of the first aspect of the invention, the method comprising coating or impregnating the absorbent article or a component part thereof with a deodorizing composition as defined in any of claims 9 to 16.

19. A use of the deodorizing composition of any of claims 9 to 16 in or on an absorbent article to reduce or eliminate odours resulting from secreted or excreted bodily fluids.
